(19) United States
(12) Patent Application Publication
(10) Pub. No.: US 2022/0287858 A1
(43) Pub. Date: Sep. 15, 2022

(54) EXPANDABLE WALL PROSTHETIC SOCKET WITH RETENTION RING

(71) Applicant: Clinton Accinni, Littleton, CO (US)

(72) Inventor: Clinton Accinni, Littleton, CO (US)

(21) Appl. No.: 17/683,294

(22) Filed: Feb. 28, 2022

Related U.S. Application Data
(60) Provisional application No. 63/154,650, filed on Feb. 26, 2021.

(51) Int. Cl. A61F 2/80; A61F 2/50; A61F 2/60; B33Y 80/00

(52) U.S. Cl. CPC A61F 2/80; A61F 2/5046; A61F 2/60; B33Y 80/00; A61F 2002/7837

(57) ABSTRACT

An expandable wall prosthetic socket configured with at least one retention ring in the socket wall and a telescoping element which interacts with the retention ring to allow the telescoping element to translate inwardly and outwardly through the socket wall, via the retention ring, such that the telescoping element is applying a force on the residual limb.

One embodiment provides for a fixed retention ring that is installed into the side of the socket wall. The retention ring is laminated into or bonded to the layered composite forming the socket wall. A telescoping element is installed through the retention and makes contact with, or is connected to, a force applicator plate. As the telescoping element translates inwardly, the telescoping element makes contact with (or pushes on) the force applicator such that the force applicator produces the load on the residual limb.

Additional embodiments provide for such features as a rotating retention ring, a two part panel with a retention ring and force applicator, and a force applicator designed into a prosthetic silicone liner.

### CROSS-REFERENCE TO RELATED APPLICATIONS

This application claims priority to U.S. Provisional Patent Application Ser. No. 63/154,650 filed on Feb. 26, 2021 and entitled “EXPANDABLE WALL PROSTHETIC SOCKET,” the disclosure of which is incorporated by reference in its entirety for all purposes.

### FIELD

Various embodiments are directed to an expandable wall prosthetic socket, and more particularly, to a prosthetic socket configured with at least one retention ring in the socket wall and a telescoping element which interacts with the retention ring to allow the telescoping element to translate inwardly and outwardly through the socket wall, via the retention ring, such that the telescoping element is applying a force on the residual limb.

### BACKGROUND

The prosthetic socket is a thermoplastic and/or laminated shell that surrounds the residual limb (stump). A common problem for prosthetic users is consistently dealing with changing pressure points on the residual limb from the socket. While the prosthetic socket is traditionally fit and modified to reduce pressure over sensitive areas such as bony prominences, this process is typically done on level ground at the clinic office or other suitable location. Socket pressures change in the socket when the position of the socket angle is changed relative to the ground during normal human activity. The change in terrain angle significantly alters the intended control modifications that have been incorporated into the socket contours that are designed to provide even socket pressure. Walking across an angled slope will cause medial or lateral displacement of the bony structures of the limb relative to the socket. Walking up or down a hill will cause anterior or posterior displacement of the limb relative to the socket wall. Athletic activity such as leaning, lifting, running, and jumping can cause large variations in socket pressures as well as the loads that are placed on the residual limb. Persistent high pressure points from the socket may cause discomfort or tissue damage resulting from the user's inability to wear the prosthetic device.

There are several methods for expandable wall sockets that currently exist.

Air Bladder Sockets. Expandable wall sockets have been used in the prosthetic industry for decades. The traditional method for making an expandable wall socket has been to fabricate a closed silicone air bladder within the interior of the prosthetic socket. The air bladder is laminated into the ridged socket wall and functions primarily as a suspension mechanism in prosthetic sockets that require a purchase over bony anatomy, such as knee disarticulation sockets and syme (ankle) amputation sockets. The flexible silicone air bladder allows the wide portion of the amputated limb to slide past the air filled bladder when donning the prosthesis. Once the residual limb is seated in the socket, the air bladder rebounds to its original shape and fills in the space above the wider bony anatomy to support the limb and suspend the prosthesis.

Mechanical Reel Sockets. A mechanical reel and a lacing system has been used in the industry to create prosthetic sockets with panels that can be lifted outward from the prosthetic socket to allow unique limb shapes through a portion of the socket. As the reel is tightened, the panel is moved back to its home position, which is the original static prosthetic socket shape.

Casting Fixtures and Modification Techniques. The use of special casting fixtures and modification techniques to the prosthetic socket are widely used to help improve skeletal stability. These sockets produce pressure on the residual limb in a more aggressive manor than a traditional socket to help stabilize the skeletal anatomy of the limb.

Motorized Socket. Another technique that is known in the prior art involves the use of a motorized socket with movable panels attached to mechanical arms and motors. The motorized socket is designed to move the panels according to data derived from sensors in the socket.

Adjustable sockets. Adjustable sockets typically integrate a mechanism that globally tightens a prosthetic socket in an effort to compensate for volume loss (fluid loss) in the user's limb during the course of the day. The entire limb is subject to the increased compression and tightening. These sockets are often an “Off the Shelf” design, and are less custom than a traditional socket.

There are several disadvantages in these existing systems as will be described next. The various embodiments of the present invention are designed to solve the disadvantages in these existing systems as will be described below.

Air Bladder sockets. The traditional method for fabricating a prosthetic socket with an expandable silicone bladder is very time consuming. The labor cost associated with fabricating the bladder into the socket is cost prohibitive. The air bladder, if damaged, is very difficult to repair and often requires the fabrication of a new socket. While the soft nature of an air bladder expandable wall socket is suitable for the suspension of a prosthetic device, it is not ideal for controlling the skeletal structures of the limb. These types of Air bladder sockets typically do not have any way to adjust the air pressure. In comparison to a mechanical system, a fraction of the force can be applied to the limb in order to stabilize the skeletal structures.

Mechanical Reel Sockets. The use of a mechanical reel and lace system in a prosthetic socket can accommodate unique limb shapes. The lace path for the reel is embedded within the socket wall. The fabrication for the socket and the lace pattern for this type of socket is fabricated over the static shape of the user's limb. That static shape represents the definitive socket shape that the reel and lace is capable of tightening down to. The lace, once installed in the socket, can be loosened by releasing the reel. Panels are cut in the socket, and the lace path runs through or within the panel, as well as through the adjacent socket wall. As the lace tightens down, the inward travel of the panel is limited to the original socket shape. The panel does not extend inward or cross the plane of the socket wall. The ability to apply force to the skeletal structures with this type of system is greatly limited. The reel and lace are prone to breakage and require maintenance several times a year to keep the system working. Another shortcoming is that the labor time and fabrication cost of producing a prosthetic socket with a reel and lace system is very high. The system requires a double lamination on the socket to encapsulate the Teflon tubing required for the lacing. Another shortcoming is that the lace tubing is sandwiched between two layers of lamination, and this doubles the weight of the prosthetic socket. Reel and lace systems are bulky and obtrusive as they can often sit one inch or more off the socket wall. The lace pattern laminated in the socket is not cosmetic and detracts from the appearance of the socket.

Casting Fixtures and Modification Techniques. The use of casting fixtures and modification techniques to control skeletal anatomy has several limitations. Casting fixtures are expensive and require special training. The use of the casting fixture and specific modification techniques creates a socket that has very aggressive contours within the final socket. These contours make it very difficult for the user to don the prosthesis (especially if there is swelling present). Once the socket is finalized, there is no ability to adjust the areas of the socket applying pressure, as they are molded into the socket walls.

Motorized Sockets. Some of the disadvantages of a motorized socket are its weight, cost, and bulk. Lightweight sockets are the preferred industry standard and most users desire the socket to be as low profile as possible. The ability to use a prosthetic socket in the water is a common requirement. An electro-mechanical socket would likely not be waterproof. The complexity of integrating motors, sensors and mechanical arms is cost prohibitive in today's prosthetic market. The declining reimbursement rates for prosthetic devices and the lack of issuance of new codes makes the probability of being reimbursed for a motorized socket highly unlikely.

Adjustable sockets. Adjustable sockets provide a system to globally tighten the prosthesis to compensate for volume loss. A significant downside of these types of systems is that volume loss typically does not occur over bony areas of the limb. While a global tightening of the limb tissue can support the skeleton by increasing the hydrostatic load on the tissue, it often creates pressure areas over bony prominences in the socket. This can lead to tissue damage and wounds on the limb. Adjustable sockets are not adapted to provide loading in specific areas of the residual limb that can tolerate pressure. Often adjustable sockets are an “Off the Shelf” design only coming in several sizes and relying on the tensioning mechanism to make the socket fit more intimate. Adjustable sockets do not provide a means to stabilize the skeletal structures of the residual limb in the socket.

Various embodiments of the present disclosure help resolve such issues in an efficient manner.

Other problems with prior art systems will be described next. Static prosthetic sockets do not accommodate or control changes in forces inside the socket. A multitude of varying forces are exerted on the user's residual limb by the prosthetic socket. Forces change in the socket depending on activity and angle the prosthetic limb is being used in. The user's residual limb experiences pressure as forces are generated by ground reaction forces from uneven ground, walking on angles or increased speed of activity. This lack of adjustability results in pain, discomfort and skin breakdown for the user. Traditionally, the only means the user has to alleviate pressure caused by the socket was to add a spacer sock designed to tighten up the socket globally. The addition of a sock increases pressure over the bony areas of the limb, typically where the pressure is occurring. Prosthetic sockets have specific areas that are weight bearing and areas that are non-weight bearing. What is lacking is the ability to simply adjust the forces being applied to the residual limb during specific activity or based on the terrain the prosthesis is being used in (in real time). Current prosthetic socket designs do not address or identify the need to be able to dynamically adjust independent areas of the socket to manage pressure on the limb during the day.

Additionally, volume loss inside the prosthetic socket does not occur in a global or uniform manner. The volume loss is specific to soft tissue areas such as the calf region. Rarely is there volume loss in bony areas. With volume loss, socket pressures change. The shape of the socket functions to distribute pressure in specific areas such as the interosseous groove, the medial tibial plateau and the popiliteal area of the limb. As volume is lost, the limb changes position in the socket and load bearing areas of the socket no longer produce the necessary pressure on the skeletal structures to stabilize the limb in the socket.

### BRIEF DESCRIPTION OF THE DRAWINGS

A more complete understanding of the various embodiments may be derived by referring to the detailed description and claims when considered in connection with the following illustrative figures:

FIG. 1 illustrates the effect on skeletal structures inside the socket when ground reaction forces are applied to the prosthesis.

FIG. 2 illustrates an embodiment of an expandable wall prosthetic socket with a retention ring that is used to help suspend the prosthesis on the residual limb.

FIG. 3 illustrates a cross-section view of an embodiment of an expandable wall prosthetic socket with a fixed retention ring.

FIG. 4 illustrates a cross-section view of an embodiment of an expandable wall prosthetic socket with a fixed retention plate.

FIG. 5 illustrates a cross-section view of an embodiment of an expandable wall prosthetic socket with a rotating retention ring. FIG. 5 also illustrates side and top views of various aspects of this embodiment.

FIG. 6 illustrates a cross-section view of an embodiment of an expandable wall prosthetic socket with a telescoping element that is fixed to a retention ring. FIG. 6 also illustrates top and side view of various aspects of this embodiment.

FIG. 7 illustrates a cross-section view of an embodiment of an expandable wall prosthetic socket with a retention ring 3D printed as an aperture in a 3D printed socket wall.

FIG. 8 illustrates an embodiment with a plurality of retention rings and telescoping element assemblies.

FIG. 9 illustrates various telescoping element and force applicator combinations.

FIG. 10 illustrates a cross-section view of an embodiment with a two-part force applicator installed in a prosthetic socket.

FIG. 11 illustrates a cross-section view of an embodiment with a force applicator designed into a prosthetic silicone liner.

FIG. 12 illustrates an exterior view of an embodiment of a prosthetic silicone liner with a force applicator.

FIG. 13 illustrates various embodiments of telescoping and retention ring combinations.

FIG. 14 illustrates a cross-section view of an embodiment with a force applicator having a specific geometry.

FIG. 15 illustrates a front view of an embodiment with an Ankle Foot Orthosis (AFO).

FIG. 16 illustrates a side view of the embodiment of FIG. 15 with an Ankle Foot Orthosis (AFO).

FIG. 17 illustrates a front view of an embodiment with a scoliosis brace.

FIG. 18 illustrates a side view of the embodiment of FIG. 17 with a scoliosis brace.

FIG. 19 illustrates a cross-section view of an embodiment integrated into orthopedic and conventional footwear.

FIG. 20 illustrates a cross-section view of the embodiment of FIG. 19 in the mid-section of the shoe.

FIG. 21 illustrates a cross-section view of an embodiment that is used in the tongue section of a shoe.

FIG. 22 illustrates an embodiment that is used in an arch support of a shoe.

FIG. 23 illustrates a top view and a bottom view of the embodiment of FIG. 22 that is used in an arch support of a shoe.

FIG. 24 illustrates a cross-section view of an embodiment that is used in a hockey skate.

FIG. 25 illustrates a side view of the embodiment of FIG. 24 that is used in a hockey skate.

FIG. 26 illustrates an embodiment that is used in a hernia truss.

FIG. 27 illustrates an embodiment that is used in a hernia belt.

FIG. 28 illustrates an exterior view of the embodiment of FIG. 5 with two retention rings.

### SUMMARY

An expandable wall prosthetic socket according to various aspects of the present invention comprises at least one retention ring in the socket wall and a telescoping element which interacts with the retention ring to allow the telescoping element to translate inwardly and outwardly through the socket wall, via the retention ring, such that the telescoping element is applying a force on the residual limb. An expandable wall prosthetic socket that allows for dynamic adjustment of socket pressures applied to a limb is provided for by various embodiments of the present invention. The system comprises a prosthetic socket configured to receive a limb. At least one retention ring in the socket wall and a telescoping element which interacts with the retention ring to allow the telescoping element to translate inwardly/or outwardly through the socket wall (through the retention ring) with the telescoping element applying a force on the residual limb. The movable element may be connected to or may make contact with a force applicator with a larger diameter than the element itself to better distribute pressure across a broader area of the limb. Various embodiments of the present invention can be used for upper extremity prosthetic sockets, lower extremity prosthetic sockets, orthopedic bracing devices, footwear applications, and various braces such as hernia belts.

### DETAILED DESCRIPTION

First, the various elements illustrated in the associated figures will be described.

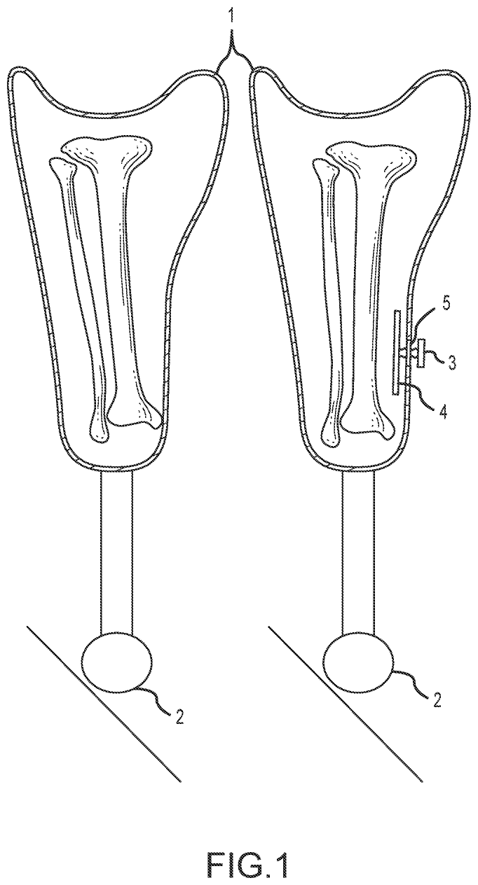

With reference to FIG. 1, an illustration is provided to show what happens to the skeletal structures inside a socket (1) when ground reaction forces are applied to the prosthesis (2). With the amputee standing on a hill the residual limb presses against the downhill socket wall in order to stabilize the rest of the body. This causes pressure and discomfort for the amputee where the bony anatomy contacts the socket wall. Various embodiments of the present invention allow the user to adjust the prosthetic socket to help stabilize the bony anatomy. By turning the telescoping element (3), which expands the force applicator (4) toward the limb tissue, the user is able to dynamically control and reallocate socket forces on the limb. Proper loading in the prosthetic socket (1) prevents tissue injury and skin break down.

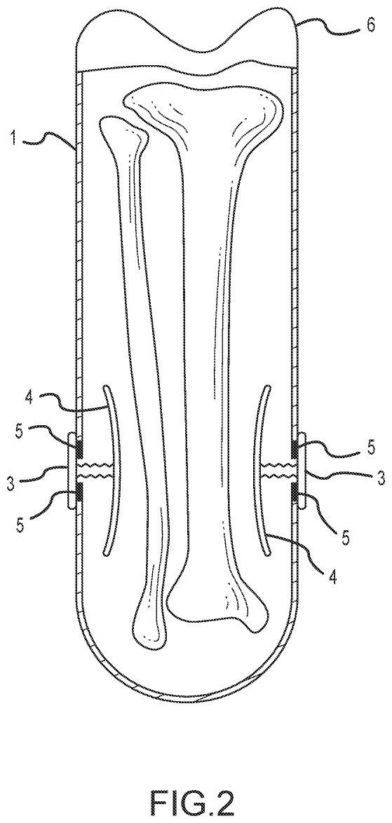

With reference to FIG. 2, an embodiment of the present invention used in a socket (1) to help suspend the prosthesis on the residual limb is shown. In amputations (types) such as symes (ankles), knee disarticulation, wrist disarticulation, and elbow disarticulation it is common practice to try to suspend the prosthesis just above the wider skeletal bony anatomy of the distal segment of the limb. Traditionally, this is done with air bladders or compressible foam, which allows the user to slide the wider portion of the residual limb in past a narrower section in the prosthetic socket. These traditional methods do not allow the user the ability to adjust the amount of suspension and often times the suspension is less than adequate. The present invention provides a significant improvement in the user's ability to fine tune the suspension of the prosthesis as well as provide a more intimate socket over unique bony structures.

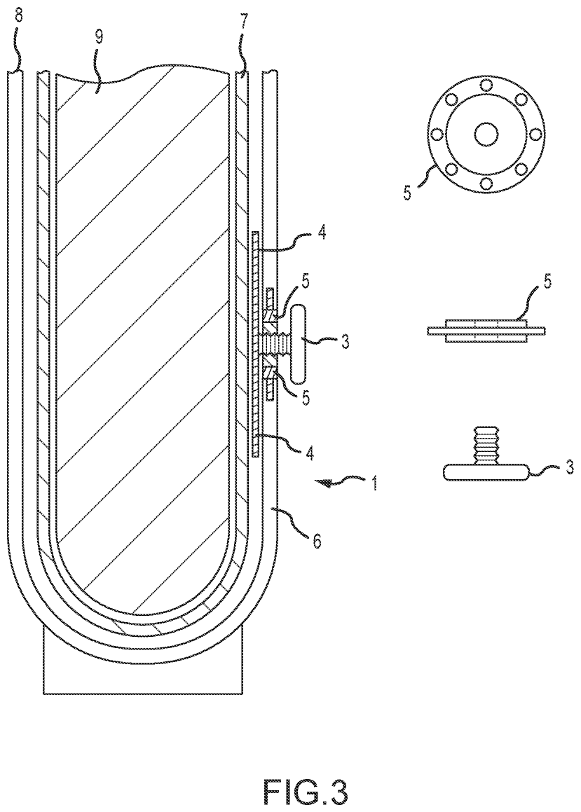

With reference to FIG. 3, an embodiment of the present invention provides for a fixed retention ring (5) that is installed into the side of the socket wall (6). The retention ring (5) is laminated into or bonded to the layered composite forming the socket wall (6). The retention ring (5) may have holes in the outer circumference section to allow the resin of the socket lamination to lock the ring into place. The aperture in the retention ring in this embodiment is threaded. The telescoping element (3) is shown threaded through the retention ring (5). The telescoping element (3) makes contact with a force applicator plate (4) that is bonded or connected to a flexible inner socket (7). The inner socket is “flexible” by nature and moves in response to pressure. The force applicator (4) is a more ridged material designed to disperse the pressure from the telescoping element (3) over a broader area. As the telescoping element (3) is disposed into the socket (1) the flexible inner socket (7) yields and the system produces a load on the residual limb (9). The residual limb (9) is illustrated inside the inner socket and may or may not be covered by a silicone or similar material.

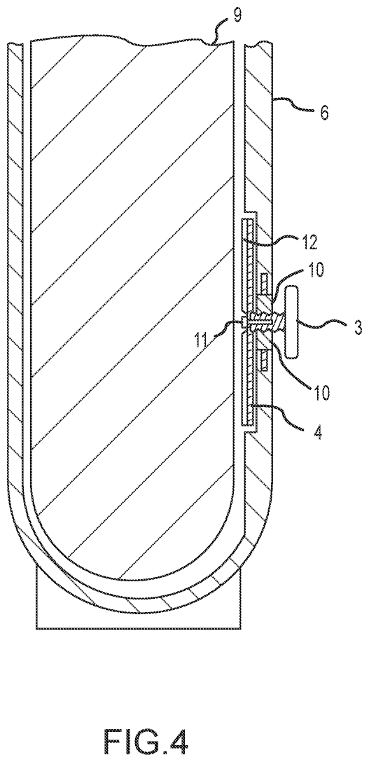

With reference to FIG. 4, an embodiment of the present invention provides for a fixed retention plate (10) that is installed into the side of the socket wall (6). The retention plate (10) is laminated into the layered composite forming the socket wall (6). The retention plate (10) may have holes in the outer section to allow the resin of the socket lamination to lock the plate into place. The aperture in the retention plate (10) in this embodiment is threaded. The telescoping element (3) is shown threaded through the retention plate (10). The telescoping element (3) is connected to the force applicator plate (4) with a screw (11) that is inserted through the back of the force retention plate (4) into the telescoping element (3). The attachment screw (11) is free to spin inside the force applicator plate (4) such that it does not rotate the force applicator plate (4) as the telescoping element (3) is moved in and out of the socket (1). The force applicator (4) may be a semi-ridged material designed to disperse the pressure from the telescoping element (3) over a broader area. As the telescoping element (3) is disposed in this embodiment it causes the force applicator (4) to produces a load on the residual limb (9) directly. The residual limb (9) may or may not be covered by a silicone or similar material. Padding material (12) is applied to the force applicator plate (4) to help protect the residual limb (9).

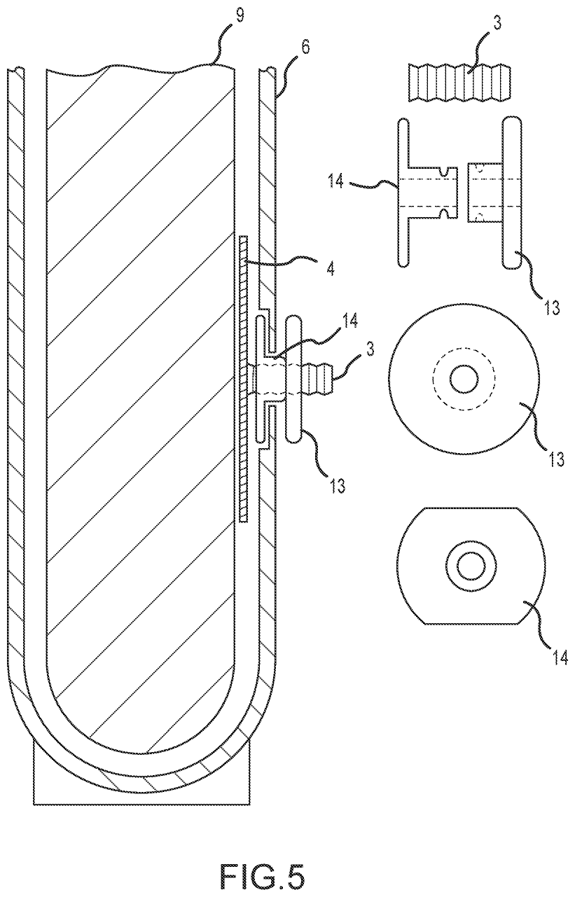

With reference to FIG. 5, an embodiment of the present invention provides for a retention ring that has a base plate (14) that is installed in the socket wall (6). The retention ring of this embodiment also comprises a rotating segment (13) which can be turned by a dial. This embodiment has a two part component featuring a front (13) and back segment (14). The back component or base plate (14) is fixed to the socket (1) such that it does not rotate in the socket wall (6). The front component (13) snaps into the back component locking the two together. The front component (13) integrates a dial and inner portion of the front component is threaded. The force applicator (4) is attached to the telescoping element (3) such that no rotation can occur between the two parts. As the dial (13) on the retention ring is turned the front component (13) rotates on the back component (14), which in turn drives the telescoping element (3), operatively connected to force applicator (4), inward and outward within the socket cavity.

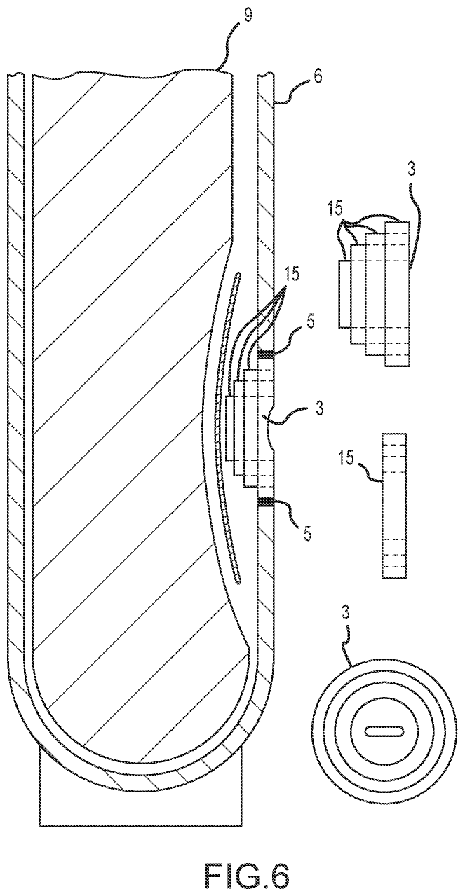

With reference to FIG. 6, an embodiment of the present invention provides for a telescoping element (3) that is fixed to the retention ring (5). The retention ring is mounted into the socket wall. In accordance with this embodiment of the present invention, the telescoping element (3) comprises a plurality of interconnected threaded sleeves (15). The innermost sleeve has a closed end and acts as the contact point for the telescoping element (3) against the force applicator panel (4). Each sleeve (15) can be threaded into the next sleeve at least half of the width of the sleeve, which creates a telescoping effect with the sleeves. When the sleeves (15) are threaded back to the home position, the sleeves rest completely inside one another, reducing the height of the telescoping element (3) to one segment. This design combines reasonable expandability with the ability to make the device as compact as possible.

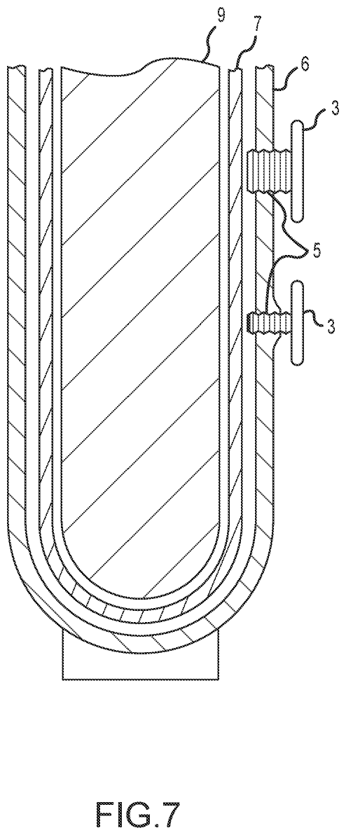

With reference to FIG. 7, an embodiment of the present invention provides for a retention ring (5) that is a 3D printed aperture in a 3D printed socket wall (6). A variety of widths can be printed to accommodate the use of various size telescoping elements. The retention ring (5) may be reinforced with additional composite material during the printing process to provide additional structural support. 3D printing is a viable option that expedites the fabrication of prosthetic socket (1). The entire assembly comprising the retention ring (5), the telescoping element (3) and the force applicator (4) can all be designed into the 3D printed socket.

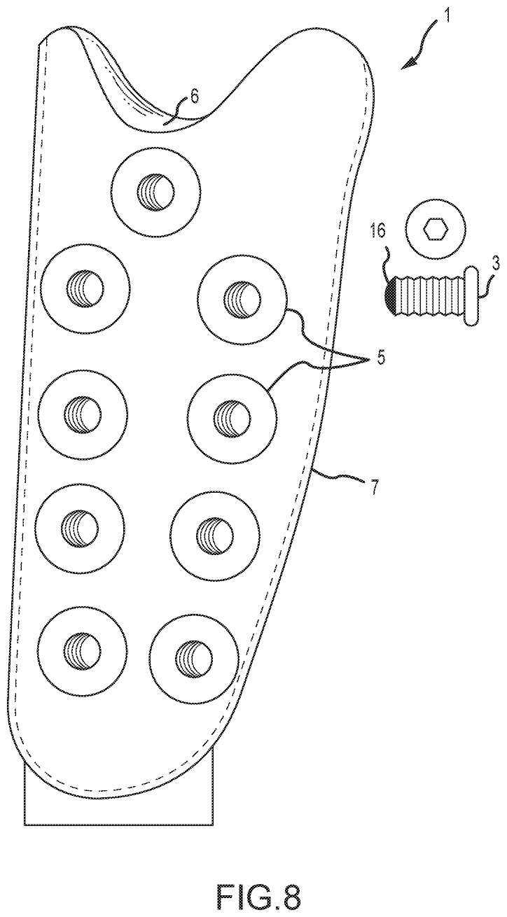

With reference to FIG. 8, an embodiment of the present invention provides for a socket (1) with a plurality of retention rings (5) and telescoping element assemblies (3). Multiple telescoping elements can be combined to create a socket (1) that is fluid with respect to the socket wall shape. This embodiment comprises a socket (1) that integrates a plurality of retention rings (5) and a plurality of telescoping elements (3) integrated into a prosthetic socket (1). Various embodiments of the present invention could incorporate a socket with a large number (ranging from two to several hundred or more) of telescoping elements (3) and adjustable force applicators, wherein the very shape of the socket (1) could be adjusted to contour to various limb shapes. These embodiments may require a sock or fabrication material comprising a large number of retention rings (5) embedded in the fabric used to form the socket wall (6), each with a telescoping element (3) which can exert a force on the residual limb. A flexible inner liner may be used in these embodiments with a large number of retention rings, in order to provide additional surface area for the telescoping elements to provide a more uniform pressure on the limb anatomy. A telescoping element (3) with a padded tip (16) could also be used with no force applicator to improve comfort when no flexible inner socket is used.

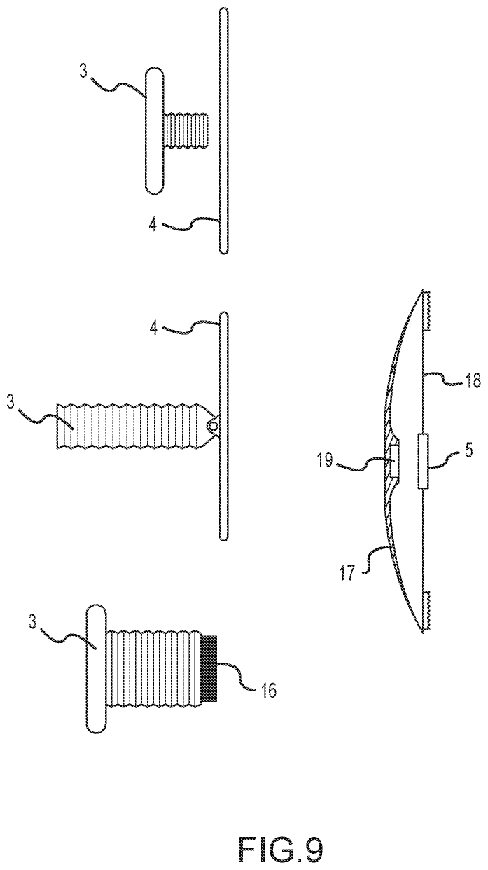

With reference to FIG. 9, various telescoping element (3) and force applicator (4) combinations are shown as will be described next. Contact Force Applicator: In this embodiment of the present invention, the telescoping element (3) is not physically attached to the force applicator (4). The telescoping element (3) makes contact with a force applicator (4) that has been bonded or attached to either a prosthetic silicone liner or a flexible inner socket. The force applicator (4) increases the surface area of the telescoping element (3). The contact force applicator is beneficial because the telescoping element can maintain a very low profile, which allows the device to be used in cosmetic socket applications. The retention ring in this configuration would be fixed in the socket (not rotating) and the telescoping element would rotate to translate the applicator inward and outward. An advantage of this design is that it would be easy for a clinician to fabricate. Attached Force Applicator: In this embodiment of the present invention, the telescoping element (3) is attached to the force applicator (4). The telescoping element (3) does not rotate in this configuration, but rather the retention ring rotates to move the telescoping element inward and outward in the socket cavity. This design can accommodate a longer telescoping element to help stabilize the skeletal structures. A good example is a trans-femoral amputee where there is a significant tissue volume and more compression is required. Split Wall Force Applicator: In this embodiment of the present invention, the force applicator and the retention ring (5) are combined into one part comprising a front plate (17) and a back plate (18). The retention ring is mounted into the ridged base plate (18) and the front plate (17) of the force applicator is a semi-rigid structure connected on its edges to the back plate (18) with a silicone type material. The silicone stretches as the telescoping element is threaded through the device allowing the front plate (17) of the force applicator to travel inward to the socket cavity and apply pressure on the limb. The device is mounted to the interior of a prosthetic socket. Velcro or other adhesives can be used to mount the device. The socket wall acts as an anchor to prevent the retention ring (5) and back plate (18) from moving in response to the load applied by the telescoping element. The retention ring could be fastened to the socket wall if desired.

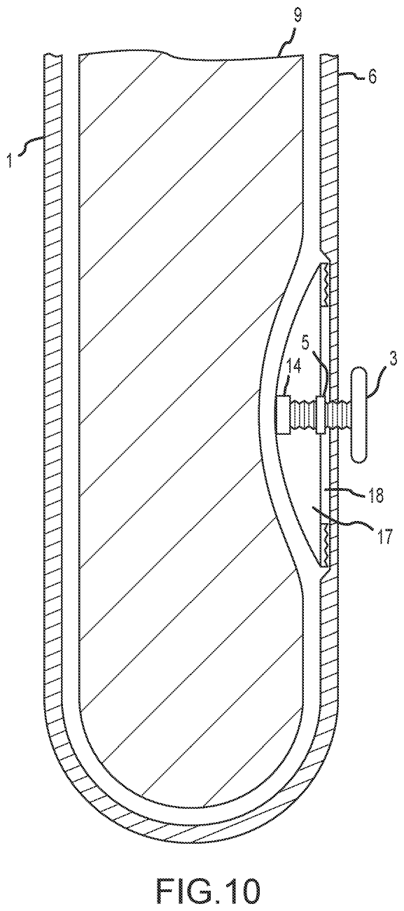

With reference to FIG. 10, an embodiment of the present invention provides for a split wall force applicator installed in a prosthetic socket. The split wall force applicator combines the retention ring (5) and the force applicator into one part. The device functions as an expandable wall module. The back plate (18) and the front plate (17) are connected (bonded) to each other on their periphery by a flexible silicone type material. The back plate (18) houses the retention ring (5) and is mounted to the socket wall (6). Velcro or other means of bonding are used to secure the back plate (18) in place along the socket wall. The telescoping element (3) is inserted through a hole in the socket wall (6) and threaded into the retention ring (5) in the back plate (18). As the telescoping element (3) advances into the split wall force applicator it makes contact with the telescoping element catch (19) (retainer) in the front plate (17) of the force applicator. As the telescoping element (3) is driven further into the applicator, the silicone material connecting the two plates expands and the front plate is allowed to translate inward.

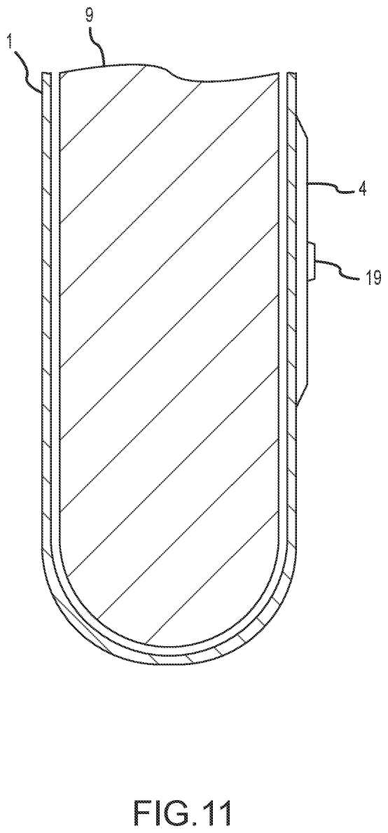

With reference to FIG. 11, an embodiment of the present invention provides for a force applicator (4) designed into a prosthetic silicone (or similar) liner. A specially designed prosthetic liner that is adapted with a semi-rigid panel that is placed in a specific weight bearing area on the residual limb (9). The force applicator (4) may have a more rigid center where the telescoping element catch (19) is located. Additionally the area surrounding the center area of the applicator may be made of a higher durometer silicone to allow some flexibility in the force applicator edges. This added flexibility allows the user to don the prosthetic liner more easily and helps to disperse pressure applied by the telescoping element by graduating the applied pressure, as the edges of the force applicator are thinner and more forgiving.

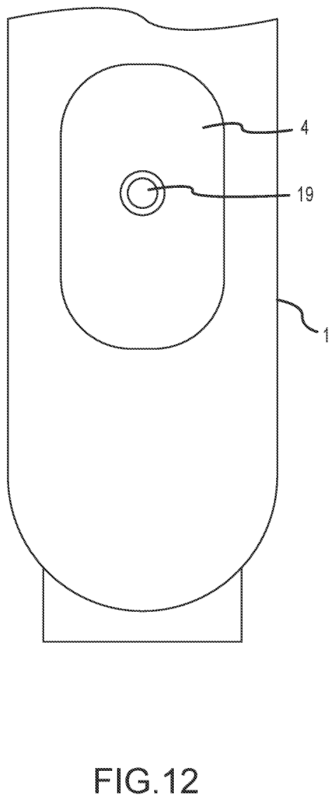

With reference to FIG. 12, an embodiment of the present invention shows an exterior view of a prosthetic silicone liner (or similar) with an integrated force applicator. The liner is better adapted to accommodate forces being applied to the residual limb. The benefit of the force applicator being attached to the liner is the socket fabrication is less complex.

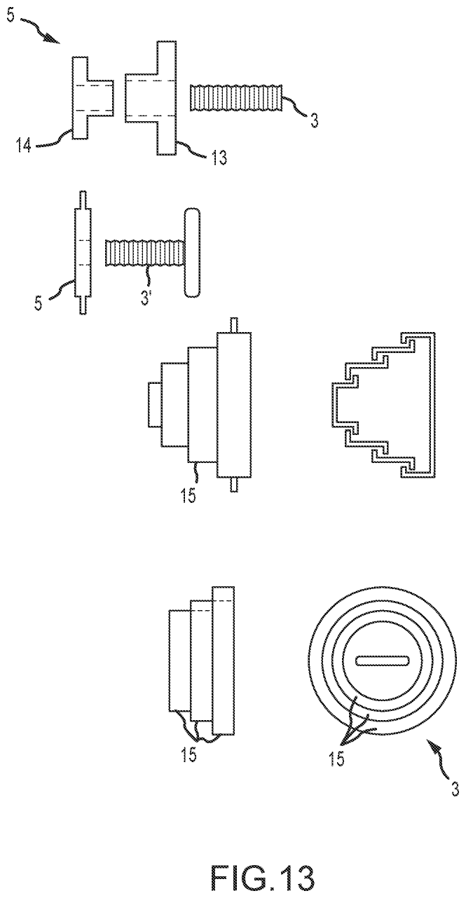

With reference to FIG. 13, various telescoping element and retention ring combinations are shown as will be described next. Rotating: This embodiment of the present invention features a retention ring (5) that is installed in the socket wall. The retention ring (5) is configured such that the outer portion (13) of the retention ring is free to rotate and the telescoping element (3) is fixed such that it does not rotate. The inner cavity of the rotating portion of the element is threaded, through which the telescoping element inserts. The rotating element (13) allows the user to advance and retract a telescoping element through the socket wall. Fixed: This embodiment of the present invention features a fixed retention ring (5) that does not rotate. The telescoping element (3) is translated through the socket in this configuration by turning the telescoping element (3) itself. A variety of known mechanical means can be used to create the ability for the telescoping element (3) and the retention ring (5) to interconnect and allow controlled movement between the two. These include but are not limited to threads, step locks, friction elements, and notched or stepped connections. Collapsible Telescoping Element with step locks: This embodiment of the present invention features a series of sleeves (15) that are inter-connected. The sleeves lock together when an interior sleeve is extended out of the interconnected outer sleeve causing a rib or step on the inner sleeve to engage in a recess formed in the outer sleeve locking the two together in a more expanded position. Collapsible Telescoping Element with Threads: In this embodiment of the present invention, the inner and outer surfaces of a series of inter connected sleeves (15) are threaded. Each inner sleeve (15) can be threaded out of the adjacent outer sleeve in order to telescope the assembly inward toward the socket center. At rest or in the collapsed position, the telescoping element (3) is at its smallest dimension, which produces minimal protrusion for the exterior socket wall. The collapsible telescoping element (3) is mounted in a fixed retention ring in the socket wall.

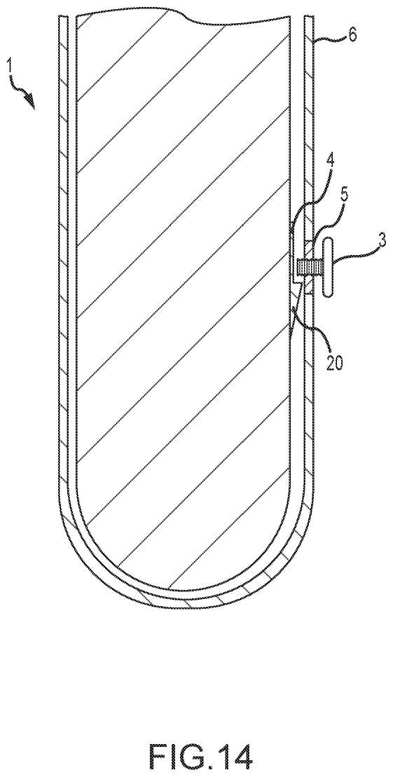

With reference to FIG. 14, an embodiment of the present invention provides for a force applicator (4) attached to a prosthetic liner or inner-socket that functions as a locking mechanism to secure the covered limb to the outer socket. In this embodiment, the force applicator (4) has a specific geometry (20) such as a lip, flange, recess or edge broad enough to interact with the telescoping element (3). After the limb is inserted into the prosthetic socket the telescoping element (3) is moved inward such that it rests on top of the geometry (20) of the force applicator (4). The residual limb is secured in the prosthesis so long as the telescoping element (3) prevents the covered residual limb from moving upward as the flange of the force applicator (4) catches on the telescoping element (3). This type of locking system is largely beneficial as component clearance is often an issue between the end of the socket and the prosthetic knee or foot. A variant of this embodiment would include a retention ring with a stepped or notched interior where the telescoping element that has the same geometry. The stepped travel of the telescoping element allows for the assembly to be air and water tight as the entire system can be covered by a rubber cover that is attached to the exterior end of the telescoping element. Simple in and out translation allows the user to operate the telescoping element without having to twist it.

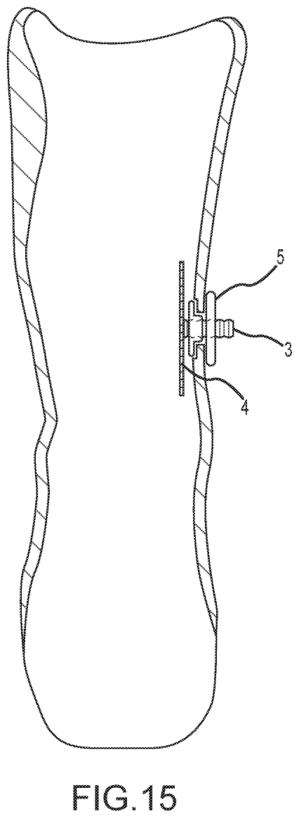

With reference to FIG. 15, an embodiment of the present invention provides for an Ankle Foot Orthosis (AFO) with the expandable wall system installed in the sidewall of the calf section of the brace. A rotating retention ring (5) is illustrated with a force applicator (4) connected to the telescoping element (3). The force applicator (4) may be padded to improve pressure tolerance for the user. The expandable wall system in an AFO device applies a load on the skeletal anatomy in a specific area, typically one of the three point pressure points designed into the orthopedic device to correct a deformity.

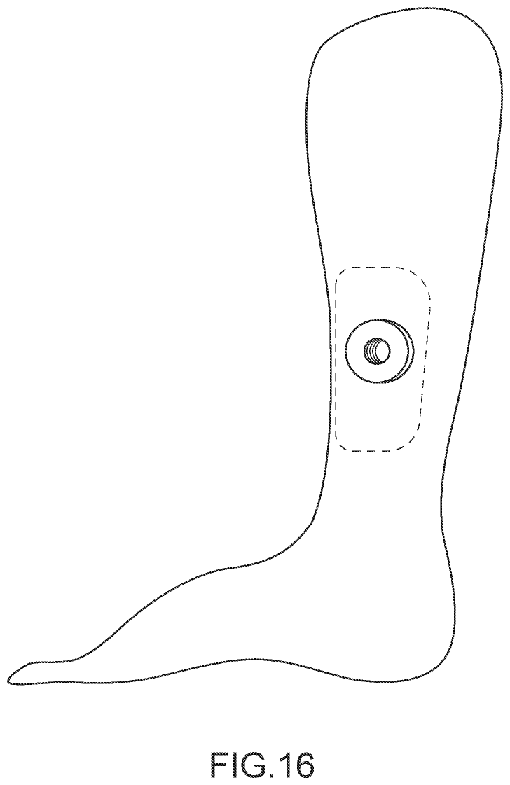

With reference to FIG. 16, the embodiment comprising an Ankle Foot Orthosis (AFO) is shown from a side view.

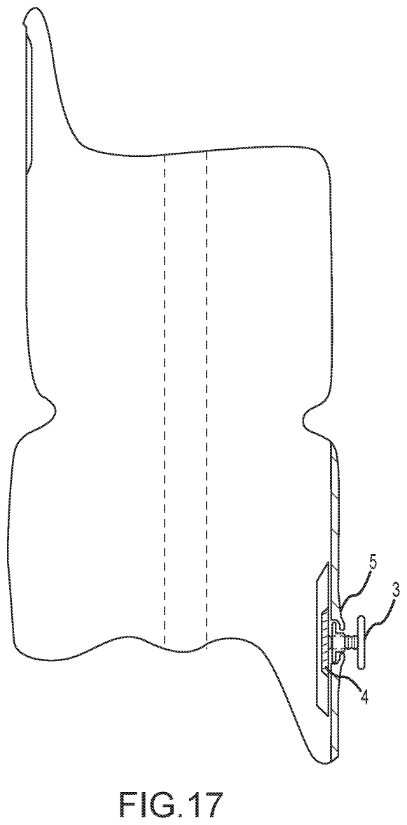

With reference to FIG. 17, an embodiment of the present invention provides for a scoliosis brace, which is a corrective spinal brace designed to reduce a curvature in the spine. This embodiment features a rotating retention ring (5) with a fixed telescoping element (3) attached to a force applicator (4) and pad (21). The scoliosis brace applies a three-point pressure system to the spine in order to change the position of the spine. In traditional braces, if more correction is needed, padding is added in one or more of the three points. The present invention allows for a means to dynamically adjust the pressure applied to the spine.

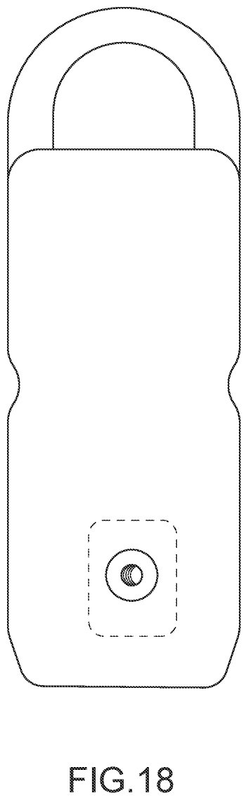

With reference to FIG. 18, the embodiment comprising a scoliosis brace is shown from a side view.

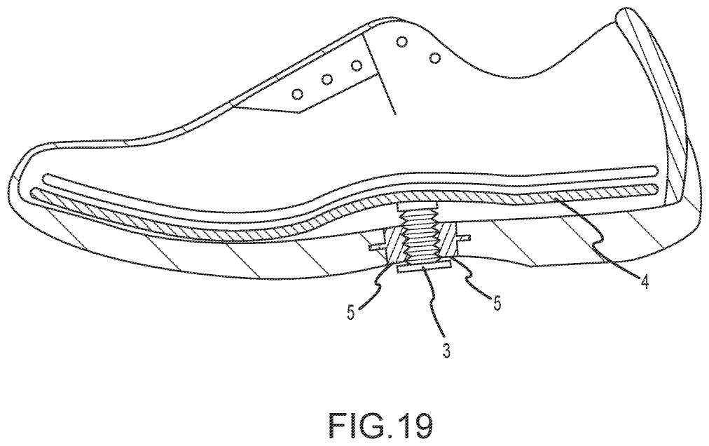

With reference to FIG. 19, an embodiment of the present invention is shown integrated into orthopedic and conventional footwear. This embodiment of the present invention comprises a fixed retention ring (5) with a rotating telescoping element (3). The force applicator (4) is an arch plate that is located under the longitudinal arch. The force applicator (4) may be padded or an additional sock liner (insert) can be placed on top of the force applicator. The retention ring (5) is molded into the arch of the external sole of the shoe. The retention ring (5) creates an aperture in the arch of the shoe to allow the telescoping element (3) to interact with the force applicator (4). Any combination of retention rings (5), telescoping elements (3) and force applicator (4) designs described in this specification could be used to achieve the purpose illustrated in this embodiment. An aftermarket installation would also be possible with a grommet type installation of the retention ring (5) into the sole of the shoe.

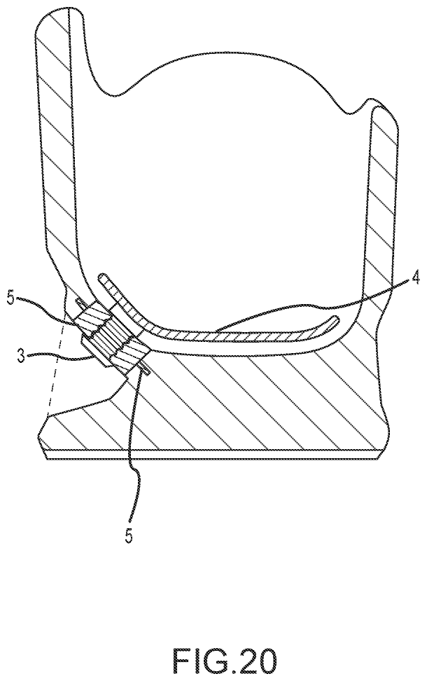

With reference to FIG. 20, the embodiment integrated into orthopedic and conventional footwear is shown in a cross section of the mid-section of the shoe. In this figure, the arch of the shoe is adjusted to a higher position.

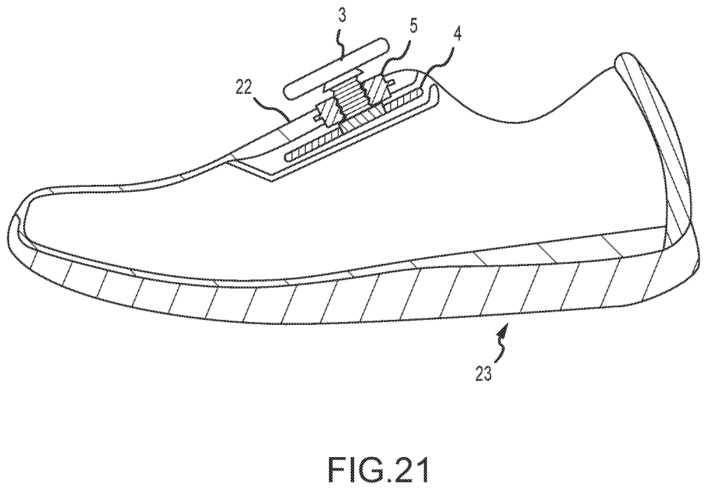

With reference to FIG. 21, an embodiment of the present invention is shown that is used in the tongue section (22) of a shoe (23) where the retention ring (5) is sewn into the outer portion of the tongue of a shoe. The tongue (22) of the shoe (23) acts in a similar functional capacity as the split wall force applicator where the back plate corresponds to the top (outside) of the tongue and the front plate corresponds to the interior side of the tongue. As the telescoping element (3) is moved through the retention ring (5) in the outer portion of the tongue the space between the outer portion and inner portion of the tongue expands helping to tighten the shoe onto the user's foot. The front plate may have a semi ridged structure or catch to accept the telescoping element. Padding lines the front plate between the foot and the plate to improve comfort on the dorsum of the foot.

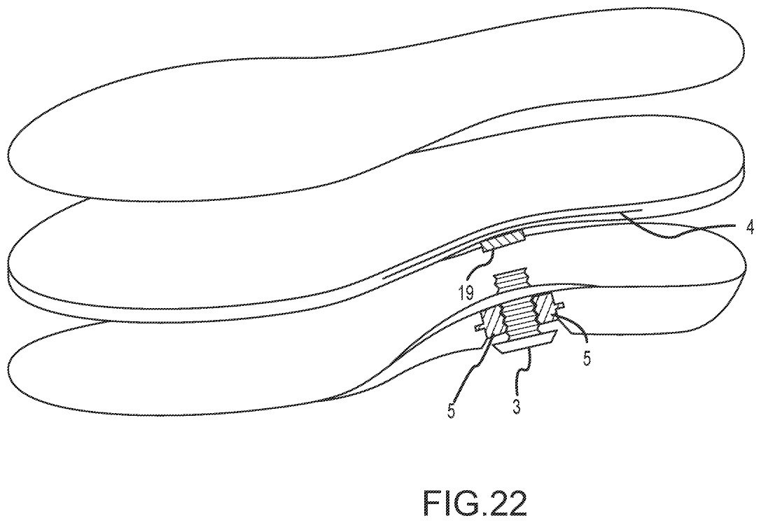

With reference to FIG. 22, an embodiment of the present invention is shown that is used in an arch support. The illustration shows a base section of the support where the retention ring (5) is installed. The base of the orthotic is a structural segment designed to support the weight of the arch as well as accommodate the retention ring for the system. The force applicator (4) (arch plate) can be a full length plate or simply a tailored piece designed to fit only in the longitudinal arch space. The force applicator (4) and sole padding can be one component or two separate pieces. The force applicator (4) has a catch (19) or receiver for the telescoping element (3) to interact with. The catch (19) protects the arch from the point pressure applied by the telescoping element (3). As the telescoping element (3) is moved inward and outward the arch support height (primarily under the longitudinal arch) is raised and lowered. This provides increased support in the arch area.

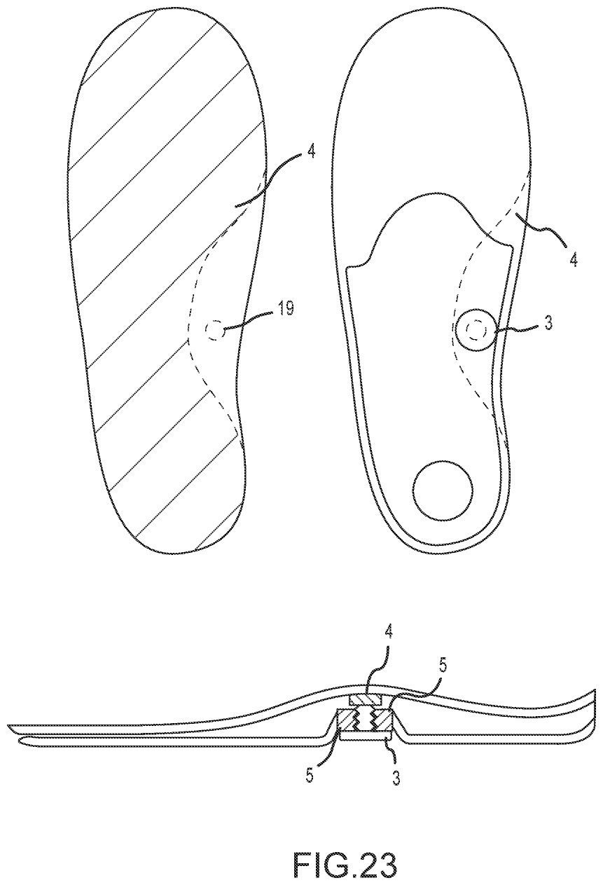

With reference to FIG. 23, the embodiment of the present invention that is used in an arch support of a shoe is shown from a top and bottom view.

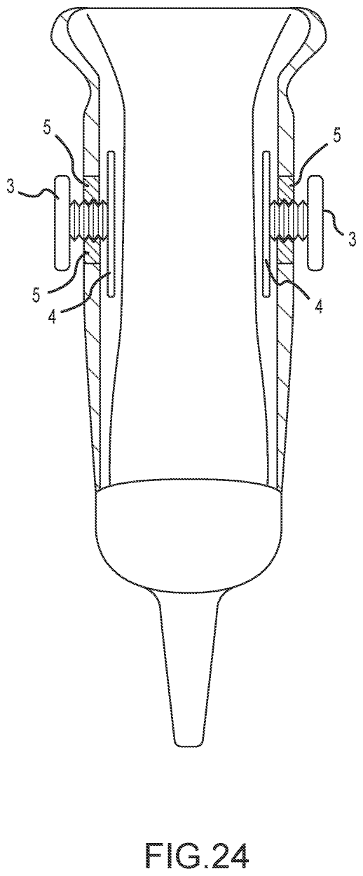

With reference to FIG. 24, an embodiment of the present invention is shown that is used in a hockey skate. This embodiment of the present invention can be used in sport specific footwear in a similar manner as in the shoe embodiment. Additional applications include the use of the device integrated into the upper boot section of athletic footwear such as a hockey skate or hiking boot, were medial lateral stability is desired. This embodiment of the present invention features a retention ring (5) sewn/molded into the upper section of the skate. The retention ring (5) is mounted in the outer portion of the skate upper. A telescoping element (3) is installed through the retention ring (5) and interacts with a force applicator (4). The force applicator (4) is contained within the padding of the interior portion of the boot. As the telescoping element (3) is moved inward and outward the force applicator (4) acts to tighten the upright of the skate providing improved ankle stability.

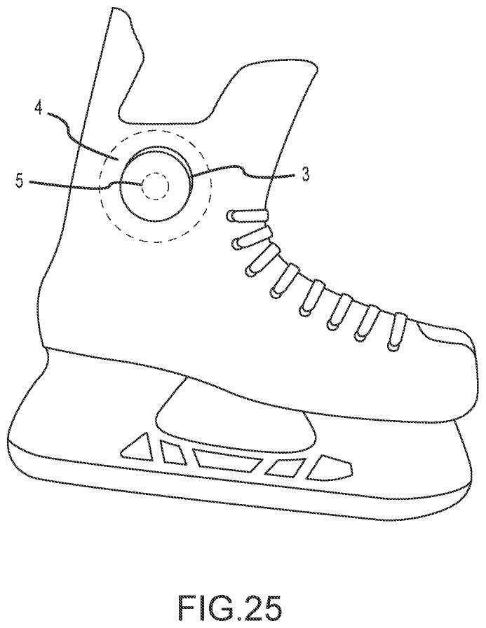

With reference to FIG. 25, the embodiment of the present invention that is used in a hockey skate is shown from a side view. In a similar manner, other embodiments of the present invention can be used in a variety of sport footwear applications such as ski boots, snow board boots, wake board boot, and the like.

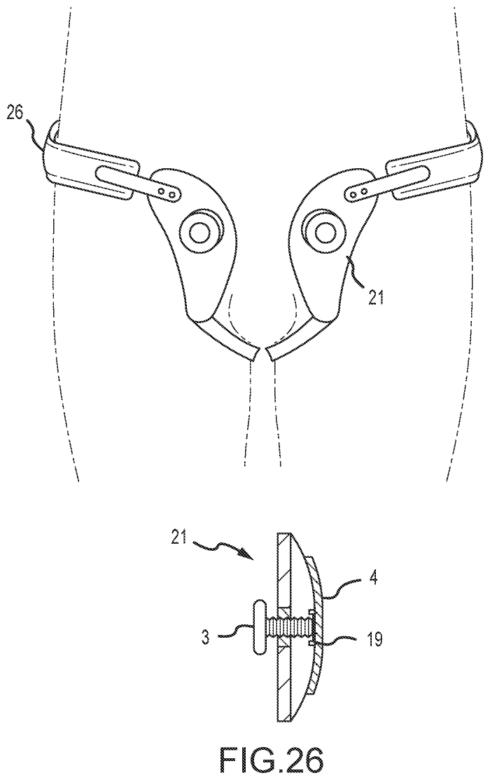

With reference to FIG. 26, an embodiment of the present invention is shown that is used in a hernia truss (metal support). In this embodiment, the retention ring (5) is mounted into the back portion of the hernia pad or pad mount. The telescoping element (3) is inserted through the retention ring (5) and interacts with a force applicator (4) contained within the pad (21). Stretchable fabric or silicone material allows the telescoping element (3) to expand the force applicator (4) away from the pad mount toward the hernia area providing support and pressure to the injury.

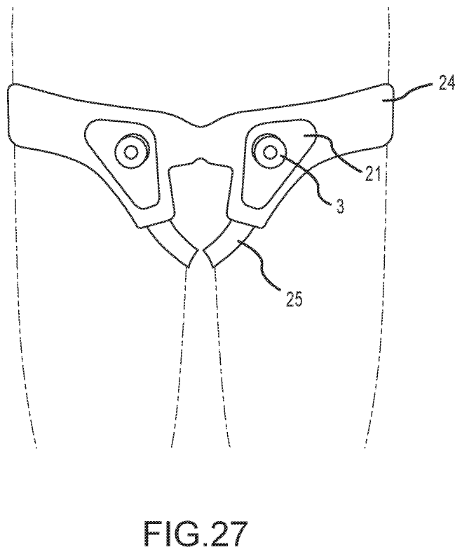

With reference to FIG. 27, an embodiment of the present invention is shown that is used in a hernia belt (24). The hernia belt (24) is similar to a hernia truss (26). The Assembly is installed into a fabric garment instead of a metal structure. The fabric on these devices is typically non-elastic. The retention ring is sewn into the outer fabric portion of the device. The telescoping element (3) is inserted through the retention ring and interacts with a force applicator contained within the pad in the device. Stretchable fabric or silicone material allows the telescoping element to expand the force applicator away from the retention ring mount toward the hernia area providing support and pressure to the injury.

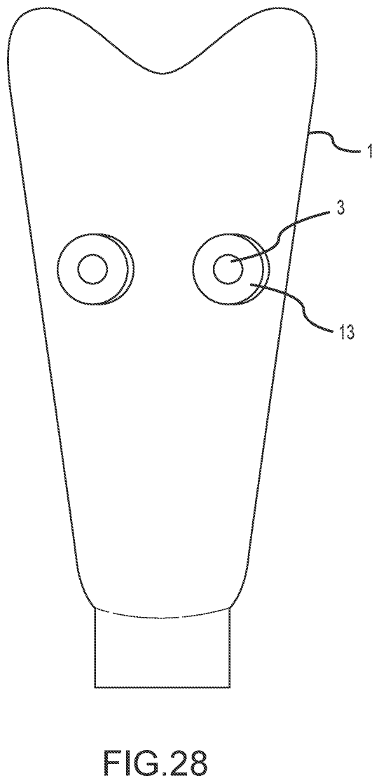

With reference to FIG. 28, the embodiment of the present invention that provides for a retention ring with a rotating segment (13) which can be turned by a dial is shown.

Exemplary System

Various aspects of the present invention detail cost effective methods and system to dynamically adjust the forces applied within a prosthetic socket. The system enables the user to change socket forces applied by the prosthetic socket to the residual limb in real time. Pressure loads can be shifted within the socket dependent on the activity or the angle that the prosthesis is being used. Various embodiments of the present invention provide methods and system to effectively redistribute pressure to different areas of the prosthetic socket as desired by the user. This will allow the user to compensate for various activities that produce different socket pressures from that of normal ambulation.

In accordance with various aspects of the present invention, the system can address volume loss by increasing the hydrostatic loading within the socket. The interior dimension of the socket can be tightened in the weight bearing areas, which provides skeletal control without applying pressure to the sensitive bony areas.

An exemplary expandable wall mechanism for use in a prosthetic socket is provided by various aspects of the present invention. The mechanism comprises a retention ring mounted in the socket wall. The retention ring acts as a fixated point of leverage in which a corresponding telescoping element interacts with the retention ring to allow the telescoping element to be moved inward and outward through the prosthetic socket wall. The telescoping element may connect or interact with a force applicator (panel) on the interior of the socket. The panel moves inward and outwardly depending on how far in or out the telescoping element is translated into the socket. The force applicator contacts the residual limb of the user, thereby acting to increase loading on a specific area of the limb which can generally accept loading/weight bearing. The telescoping element can be directly connected to the force applicator in order to suspend the applicator within the socket assembly. Alternatively, the telescoping element can simply contact a force applicator that is adhered to an inner socket or prosthetic liner with an integrated force applicator. The force applicator provides a surface such that the pressure generated by the telescoping element to the residual limb is spread out over a larger area. The retention ring mechanism can be an individual component that is installed or fabricated into the socket wall or a 3D printed structure that has a mechanical feature such as threads, steps, spiral steps, notches or a friction interface to provide controlled travel of the telescoping element through the retention ring aperture while maintaining the position and depth of the telescoping element within the ring. The more precise the movement between the retention ring and the telescoping element, the finer the adjustment that can be made with respect to the travel of the panel acting on the residual limb. For example, threads offer a finer travel range of adjustability, wherein a notched or stepped connection between the retention ring and post may offer degrees of travel closer to ⅛″ per “click”.

In accordance with various embodiments of the present invention, a mechanical technique is used to create an expandable wall within the prosthetic socket. Various aspects of the present invention use a retention ring, a telescoping element, and an optional force applicator. The present invention takes advantage of the ridged socket wall (or a socket strut if the socket is a non-continuous design) to act as a leverage point. The retention ring is connected to, or is in contact with, the ridged socket wall. The telescoping element can be moved through a retention ring, which mechanically interacts via threads, friction, step-locks, and the like, with the telescoping element, thus controlling the position of the telescoping element within the retention ring. As the telescoping element is driven into the socket interior, the telescoping element contacts and moves the force applicator inward toward the residual limb. The force applicator applies pressure to a specific weight bearing or suspension area of the limb, which helps to control the skeletal structures of the limb.

It will be appreciated that various aspects of the present invention provide for several advantages as will be described next.

Various embodiments of the present invention allow the user to adjust the pressures applied by the socket in real time, resulting in an improved skeletal stability in the socket. Socket forces change depending on the terrain or amputee's activity that the prosthesis is being used on. For example, when descending a hill, the user extends the residual limb in the socket in order to slow the ambulatory decent rate. This extension force causes the distal anterior aspect of the tibia bone to move forward in the socket such that the bone makes firm contact with the socket wall, which can cause tissue injury to the use. Various aspects of the present invention allow the user to apply corrective pressure to the front loading portions of the prosthetic socket. This will counteract the forward motion of the tibia, caused by the ground reaction forces, that is a result of the descent down the hill. The user can adjust the telescoping element in the socket accordingly to resist movement of the bone shaft toward the socket wall, therein providing skeletal stability to the residual limb during that specific application or activity.

Various embodiments of the present invention provide a significant mechanical advantage, and this mechanical advantage is used to exert force to the skeletal structures of the limb. The direct relationship between the telescoping element and the force applicator allow the user to expand the force applicator into the interior of the socket with relative ease simply by turning the dial on the telescoping element (or rotating retention ring). The direct path of the telescoping element applies a perpendicular force to the applicator, which correspondingly moves the limb tissue and skeletal structures in the socket to a corrected position.

Various embodiments of the present invention provide for a system that can be used to apply pressure to specific areas of the socket to stabilize the skeletal structures of the limb. Various aspects of the present invention can be installed in different regions of the socket, often in load bearing and weight bearing areas intended to support and control the skeletal structures of the limb. Unlike adjustable sockets, which feature a global tensioning system and are intended to compensate for volume loss, the present invention applies strategic pressure in different places.

Various embodiments of the present invention provide for a system that is low profile and less obtrusive. The height and width of various embodiments of the present invention can be made to be very low profile. In accordance with an embodiment of the present invention, the retention ring comprises a threaded aperture that can be almost entirely accommodated within the thickness of the socket wall. The telescoping element can also be flush with the socket wall depending on how deep the telescoping element has been extended into the socket wall. The length of the telescoping element can vary, such that at rest the element protrudes from the socket wall, but as the element is moved inward (to its final desired position) it becomes flush with the exterior portion of the retention ring.

Various embodiments of the present invention provide for a system that allows for an unlimited amount of tensioning/control. In accordance with an embodiment of the present invention, the telescoping element of the device can be designed to any length. A longer telescoping element can be used in the system to provide increased range and movement of the force applicator into the socket chamber. Different amputation types, such as trans-femoral and variations in tissue density may require longer telescoping elements in order to move and compress the limb tissue to stabilize the skeletal structures. In accordance with various embodiments of the present invention, the telescoping element may be made of a plastic material such that the element can be cut to a custom length by the clinician.

Various embodiments of the present invention provide for a system that is low cost. The low cost makes the system accessible to a broad range of patients. The device can be installed into a prosthetic socket with minimal additional labor cost. Additionally, many parts of the system can be reused in subsequent socket installations.

Various embodiments of the present invention provide for a system that utilizes a simple design that is very durable. The expandable wall system includes three components: A retention ring, a telescoping element, and a force applicator. The simple mechanical design limits the potential of failure or breakage of the system. Each component of the system can be replaced independently.

Various embodiments of the present invention provide for a system that produces a broad range of pressure adjustability within the prosthetic socket. The device can range from no pressure on the skeletal structure to a maximum pressure determined by the length of the telescoping element. The invention when placed in the home position (no pressure) does not enter or distort the interior socket space. This makes it easier for the user to apply the prosthesis or remove the prosthesis, as the interior dimension of the socket is not changed. As the socket is tightened, the insertion of the user's residual limb into the socket becomes more difficult due to the hydrostatic loading the expandable wall applies to the limb. This same principle applies to removing the prosthesis. There is a significant advantage to be able to apply pressure to the skeletal structures of the limb after it has been inserted in the socket.

Various embodiments of the present invention provide for a system that can be used to suspend the prosthesis. Various aspects of the present invention can be used to suspend the prosthesis to the residual limb. In unique amputation types, such as knee disarticulations and syme (ankle) amputations, the distal bony anatomy is more broad than the proximal bone structures. The expandable wall of the present invention is used to create a purchase or tighten the socket just above the broad skeletal geometry, which maintains suspension of the prosthesis on the limb. Various aspects of the present invention can also be used to suspend the prosthesis through the use of a specially designed force applicator that has a wedge shape built into the applicator. In accordance with various embodiments of the present invention, the force applicator is bonded or attached to a prosthetic liner (silicone or equal) used to cover the residual limb or an inner socket that is sealed to the users limb (this is typically done with a sealing sleeve). As the covered limb is inserted into the outer socket (which houses the retention ring and the telescoping element), the force applicator aligns with the telescoping element aperture in the socket wall. As the telescoping element is extended into the socket to a fixed depth, the element rests proximal to the wedge geometry of the applicator. This prevents the force applicator from being able to slide back out of the socket, resulting in suspension of the prosthesis to the limb. The retention ring and the telescoping element may be installed in the socket such that the angle in which the telescoping element interacts with the force applicator is a more downward angle (rather than perpendicular), so that when the telescoping element is tightened it pushes the force applicator and the residual limb downward into the socket. A downward force into the socket would help secure the connection between the limb & liner or inner socket) and the outer socket by mitigating all movement between the two.

Various embodiments of the present invention provide for a system that can be installed into a socket post fabrication. The ease of use of the system allows a clinical professional to install the system into a socket after the socket fabrication has been completed. This saves money and clinical time for the prosthetic clinic and offers the amputee a viable solution to continue using a prosthetic socket that no longer fits. A common problem that amputees often face is volume loss, especially after having undergone an amputation surgery. The residual limb shrinks quite rapidly. As soon as some volume loss has occurred the clinician can install the retention ring into the socket wall simply by drilling a hole through it and inserting the retention ring. The force applicator is installed on the interior of the socket and connected to the telescoping element. This allows the user to tighten the socket in the necessary areas to ensure the socket fit remains intimate as the limb naturally loses volume and the swelling subsides. This ability to keep weight bearing areas tight and bony areas relieved helps prevent tissue injury and pain for the amputee.

In accordance with various embodiments of the present invention, a system is provided for that uses a mechanical assembly to apply pressure to specific weight bearing areas of the socket. The mechanical assembly is adjustable in real time and allows the user to change the forces in the socket to stabilize the skeletal structures of the residual limb depending on the ground terrain or activity the user is engaging in. One socket can integrate from one of these mechanical assemblies to many assemblies all within the same socket. As described above, prior art solutions include modification techniques that apply pressure to skeletal structures but are not adjustable. Some prior art solutions utilize “adjustable prosthetic sockets”, however, these prior art solutions address volume loss in the residual limb, which focuses on a global tightening of the socket structure, and this tightens the entire socket vs. specific areas of the limb that are intended to accept loading and pressure.

Various embodiments of the present invention provide for a system that allows for a dynamically controlled mechanical expandable wall socket system, which allows the prosthetic user to independently adjust each force applicator beyond the plane of the socket wall within in the prosthetic socket. The force applicator stabilizes the skeletal structures of the residual limb improving socket control and performance of the prosthesis.

Various embodiments of the present invention will be described next.

Fixed Retention Ring with rotating Telescoping Element. This embodiment features a fixed (non-rotating) retention ring that is installed, molded or 3D printed into the socket wall. A simple example is a threaded aperture through the socket wall. A telescoping element (in this case a threaded post) is threaded through the retention ring and is in connection with the force applicator on the interior of the socket. The post may be anchored to the force applicator or it may simple contact the force applicator. Downward pressure of the limb in the socket will push the force applicator back toward the socket wall if the telescoping element is backed out. The retention ring would most likely be tensioned such that the post would not spin freely and the setting or amount of travel desired by the user would not change unless the user manually adjusted the post.

Rotating Retention Ring with fixed Telescoping Element. This embodiment features a retention ring that can be configured such that it rotates within the socket wall, using a dial such that the user can rotate the retention ring to move the telescoping element (threaded post) inward and outward. In accordance with this embodiment of the present invention, the telescoping element is fixed to the force applicator such that the telescoping element does not rotate. As the retention ring dial is rotated, the telescoping element (operatively connected to the force applicator) moves inward or outward depending on the direction the dial is turned. A tensioning mechanism may be added to the dial to maintain the position of the telescoping element.

Telescoping Mechanism mounted to the Retention Ring. In accordance with this embodiment of the present invention, the telescoping element can be a series of inter-connected components, wherein each component fits within the next component with locking tabs, where the smallest cup/sleeve can be depressed and by twisting it will lock within the adjacent cup. This will allow a depressed position to be maintained and a specified amount of travel as the telescoping element, which acts to operatively move the force applicator inwardly that same amount. This embodiment is similar to a stack of cups analogy, relatively flat when they are inside each other, however, when stacked the opposite way or expanded out they are much longer, each interlocking to maintain the height of the stack. The telescoping element could also feature threads on the contacting surfaces of the sleeves or cups. This configuration allows each cup to be threaded a set distance into the next cup allowing for the series of sleeves/cups to telescope inward or outward to move the force applicator.

Fixed panel with Retention Ring. In accordance with this embodiment of the present invention, a two-part panel is provided, wherein the outer panel (faces limb) is connected to an inner panel (adjacent to the socket). The panels are connected by an elastic material on their periphery. The inner panel is attached to the socket wall and contains the retention ring. The retention ring may be connected to the socket by mechanical means. The telescoping element is inserted through the socket/device wall and threads into the inner panel. The retention ring in the inner panel secures the telescoping element. As the telescoping element is moved into the socket it makes contact with the outer panel and results in translation of the outer panel toward the interior of the socket applying pressure to the residual limb. The inner panel may be bonded or fastened to the socket wall.

Retention Ring and Telescoping Element (Basic). In accordance with this embodiment of the present invention, the retention ring and post are used to apply a force to the residual limb directly without a connecting force applicator panel. A flexible inner socket may be used between the limb and the telescoping element/retention ring as an interface helping to expand the load bearing area. Additionally, the telescoping element and retention ring design may also be used to interact with the limb directly if there are a larger number of posts (to decrease point pressure) or they may interact with a silicone liner (or similar material worn over the residual limb) with a reinforced area on the liner where the post and retention ring will apply a force to the limb and liner.

Plurality of Telescoping elements. In accordance with this embodiment of the present invention, multiple telescoping elements can be combined to create a socket that is fluid with respect to the socket wall. With reference to the various figures, a socket is illustrated that integrates one to three expandable wall segments integrated into a prosthetic socket. In accordance with an aspect of this embodiment, a socket can be provided with a large number of telescoping elements and adjustable force applicators, or a just a plurality of telescoping elements (e.g., 100), wherein the very shape of the socket could be adjusted to contour to various limb shapes. A sock or material with a large number of retention rings embedded in the material can be used to form the socket wall, each with a telescoping element which can exert a force on the residual limb. A flexible inner liner may be used in this application to provide additional surface area for the telescoping elements to provide a more uniform pressure on the limb anatomy.

Component variations will be described next.

Retention Ring Variants. The retention ring can use a variety of mechanical designs to control the telescoping elements movement through the aperture of the retention ring. Some possible options are:

Friction: The retention ring creates friction on the telescoping element that prevents unwanted movement of the telescoping element inward or outward. O-rings, a split thread with a compression wedge, metal apertures that are at an alternate angle compared to the primary aperture where the telescoping element is inserted are examples.

Threads: A threaded retention ring. In this variation, the telescoping element also has corresponding threads.

Ratchet, geared or stepped design: In this variation, the telescoping element has elevated ribs that correspond with similar ribs on the interior of the retention ring. This allows for clicks that equal inward or outward movement of the telescoping element.

3D Printing: In this variation, 3D Printing the Retention Ring may be a 3D printed aperture in socket (for example—a threaded boss) or a separate part installed in the socket wall.

Telescoping Element Variants:

Friction: In this variation, a smooth post that passes through a friction mechanism inside the retention ring. This could be a series of O-rings, close tolerances in the material, or mechanical friction spring.

Threaded telescoping element: In this variation, a threaded post that is moved inward and outward of the socket wall by turning either the post itself (fixed retention ring) or by turning the retention ring (fixed post).

Stepped, Geared or Ratchet: In this variation, a post of a defined length that has a series of notches or steps where each notch defines a set distance. As the element is moved inward and outward in the retention ring it moves a “set distance” defined by the size of the step or notch as it passed through a fixed retention ring.

Telescoping Sleeves/Cups: In this variation, a series of inter connected sleeves/cups where the outside of the inner sleeve threads into corresponding threads on the inside of the interfacing outer sleeve. Alternately, the sleeves may connect with a stepped locking mechanism that locks or snaps into place as the sleeve is extended within the adjacent sleeve. With the inner sleeve having a raised portion and the outer sleeve having a recess or receptacle for the raised geometry. The inner sleeve in both of the telescoping sleeve embodiments would have a closed end, which interfaces with the force applicator portion of the system.

Force Applicator Variants:

Attached to telescoping Element: In this variation, the force applicator is operatively connected to the telescoping element. As the telescoping element is moved inward and outward in the socket, the force applicator moves accordingly. The benefit of this configuration is that the force applicator is suspended in the socket by the telescoping element. The present invention can installed in a socket where there may not be additional coverings on the residual limb such as a silicone liner or a flexible inner socket, the force applicator can be applied directly to the residual limb. The connection between the telescoping element and the force applicator allow the applicator panel to be retracted by adjusting the telescoping element.

Attached to an inner socket: In this variation, the force applicator can be attached to an inner socket, where the force applicator is operatively connected to the inner socket. The telescoping element is not operatively connected to the force applicator in this case. Suspension of the force applicator is achieved by the applicator being mounted to an inner socket. The telescoping element applies pressure to the force applicator simply by moving the telescoping element inward and out ward. This design relies on the flexible inner socket or the movement of the limb to retract the force applicator.

Split Wall Force Applicator: In this variation, a force applicator that features two connected panels. The design includes a base plate and an expandable wall segment that is attached to the base plate by means of a silicone type material. The entire assembly in mounted on the interior of the prosthetic socket. The base plate features a built in retention ring where the telescoping element is inserted through. The expandable wall segment has a element cup designed to receive the telescoping element. The telescoping element is inserted through a hole in the socket and engages with the retention ring. As the telescoping element is feed through the retention ring the telescoping element makes contact with the element cup. As the telescoping element is moved through the retention ring the more the expandable wall segment moves inward. In this embodiment the retention ring would not need to be connected to the socket wall, but does use the socket wall as support to maintain the retention ring in position. The base plate of the split wall force applicator would be bonded or attached to the socket wall by means of Velcro or adhesive.

Prosthetic Liner with Embedded Force Applicator: In this variation, a prosthetic liner is configured with an integrated force applicator designed to interact with the telescoping element. The liner has a force applicator (semi rigid structure) molded into the silicone in a specified load bearing area. The structure acts as a way for pressure distribution as the telescoping element is extended inwardly into the socket.

Force Applicator Designed to be Bonded to Prosthetic Liner: In this variation, a prosthetic liner is designed where the force applicator is bonded onto the liner. The applicator is bonded to the exterior of the prosthetic liner during the fitting process. The advantage of having the force applicator bonded to the liner is a simplification of the fabrication process to build the expandable wall system into the socket by eliminating the need to install the force applicator into the socket assembly.

Force Applicator as a Locking Mechanism: In this variation, the force applicator is bonded to the either the silicone liner or an inner socket. The shape of the force applicator is such that there is a wedge shape that provides a suspension surface for the telescoping member to engage with. As the telescoping element is advanced inwardly it creates an area where the wedge shaped force applicator is locked into the socket. Alternatively, the force applicator may simply have a recess where the telescoping element extends into the recess to lock the two together.

Various applications of the present invention will be described next.

Orthopedic Bracing: Various aspects of the present invention have similar uses in orthopedic bracing applications wherein the same configuration of a telescoping element, retention ring and a force applicator are installed in the wall or structure of an orthosis. To a large extent, orthopedic bracing applications or orthotic devices rely on a 3-point pressure system to correct a weak or deformed limb or segment of the body. The system in these applications would be installed in the wall or structure of the orthosis in one or more of the necessary three point pressure areas required to correct or support the extremity. The adjustability of the system improves the effectiveness of the orthosis during the initial correction process. Various aspects of the present invention can provide continued adjustability of the device as the deformity becomes more malleable as a result of the corrective process, allowing for further correction beyond the original goal.

AFO: Movable wall integrated within an Ankle Foot Orthosis (AFO): In accordance with an embodiment of the present invention, a retention ring is installed in the sidewall of an AFO device. The telescoping element is installed through the retention ring and connects to the force applicator on the interior of the AFO structure. As the telescoping element is tightened the force applicator is moved toward the lower leg pushing on the extremity above the ankle joint. The force operatively changes the position of the extremity inside the AFO Device. The system creates the opportunity to dynamically control the corrective force applied by the AFO. The expandable wall system can be configured in all of the embodiments described for the device referenced the prosthetic socket application.

TLSO: Scoliosis application Movable pads: In accordance with an embodiment of the present invention, the expandable wall system can be applicable to spinal bracing. With reference to the figures, the integration of the expandable wall system into a scoliosis TLSO brace is illustrated. Typically, there are specific pads placed inside the TLSO designed to apply pressure to the spine in order to correct the deformity. The retention ring is installed in the sidewall of the spinal brace where a pressure point is designed into the device. A force applicator can be attached to the corrective pad and connected to the telescoping element, which runs through the retention ring. As the telescoping element is moved inward or outward the pressure applied by the force applicator is increased or decreased. The expandable wall system can be configured in all of the embodiments described for the device referenced the prosthetic socket application. Spinal Extension Braces such as a Cash or Jewett brace apply a three point pressure system to the spine in the sagittal plane. The expandable wall system could be installed on any of the three pressure pads of the brace.

Fracture Bracing & Casting: Corrective Force Application: Fracture bracing uses similar three point pressure system principles coupled with hydrostatic loading of the limb such that the surrounding tissues are compressed and the affected bone is supported. In accordance with an embodiment of the present invention, the retention ring is installed in the side of the fracture brace opposite the fracture to provide mild corrective pressure to the fracture site. In accordance with this embodiment of the present invention, the system uses a force applicator coupled with the telescoping element running through the retention ring to adjust the amount of pressure being applied to the fracture site. Additionally, this embodiment of the present invention could be installed within a fracture cast, where the retention ring is embedded in a fiberglass (or similar) cast sock or where the retention ring is tied in during the casting process. The expandable wall system can be configured in all of the embodiments described for the device referenced in the prosthetic socket application.

Knee Brace: Suspension element or load shifting element. Knee braces often apply a 3 point pressure system to the knee in the frontal plane. The force is applied at the center of the joint from the medial or lateral aspect of the knee in order to open up the joint space on the opposite side of the joint. In accordance with this embodiment of the present invention, the knee brace is adjusted though a mechanical expandable wall assembly located at the knee joint or one of the other three point pressure areas. The retention ring located in the brace joint is aligned with the center of the anatomical joint through which a telescoping element is installed. The element acts on a force applicator located on the inside of the brace joint allowing for pressure to be applied to the anatomical joint itself. Alternatively, this embodiment of the present invention can be used to help improve the suspension of knee orthosis. Maintaining a knee brace in position is typically problematic as the thigh is larger than the calf and the device tends to migrate downward during activity. The expandable wall system can be integrated into the upper assembly of the knee brace where the retention rings are installed in the brace uprights above the knee. The telescoping element would translate through the retention ring and act on the force applicator (panel) to gain a purchase above the anatomical boney structures of the knee (just above the condyles of the knee).

Hernia Belt/Truss. A hernia belt or truss can be modified to incorporate the expandable wall into the pad system. In accordance with this embodiment of the present invention, a truss structure with pads is aligned/positioned over the hernia site. The retention ring is mounted on the ridged backside of the truss/pad with the telescoping element operating through the retention ring. The force applicator is built into the interior of the pad and can be adjusted inwardly or outwardly to apply additional pressure on the herniated area. The adjustable panel could also be installed in a garment or belt system where the panel system would work in a similar fashion.

Footwear and Arch Supports. In accordance with various additional embodiments of the present invention, a system provided that can be used in arch supports and footwear. The control and stabilization of the skeletal structures in the foot is the foundation of all orthopedic bracing. Typically, arch supports and orthopedic (and non-orthopedic) footwear are used to support and align the bones in the foot to influence the alignment of the knees, hips, back and spine. Corrective measures taken with arch supports and footwear can resolve headaches, knee degeneration issues, hip pain, back pain and a variety of other medical problems. Those skilled in the art of orthoses and orthopedic bracing are familiar with a multitude of techniques and modifications that can be done to either a foot orthoses or to the patient's footwear to help correct a deformity or support a weak joint. While these modifications are successful in most circumstances, they require a medical professional trained in the art to facilitate the correction. These devices and the required modifications are not adjustable by nature. To a large extent the required modification on footwear and arch supports is properly determining the correct arch height. Raising and lowering the longitudinal and transverse arches to achieve an optimum alignment of the foot and ankle complex is typically done by adding padding or removing material from the support/shoe. Various embodiments of the present invention provide a system to raise and lower the arch with a simple mechanical assembly, which can exert enough mechanical leverage to lift the arch and maintain the adjustment under the load of the user's body weight. The ability to adjust the arch height at any time provides a significant benefit to the user. Depending on the activity or terrain the user is engaged with, it may be necessary to increase support from the device. Fatigue and over use is another problem that may be solved by various embodiments of the present invention. For many athletes and users who are on their feet for extended periods of time, the muscles in the foot become fatigued and the arch of the foot collapses to a degree. The ability to add additional support helps the user continue the activity without risk of damaging the structure of the foot and enabling the user to continue more comfortably and with less pain.

Footwear. In accordance with various embodiments of the present invention, orthopedic footwear, standard footwear and sport specific footwear applications are relevant for the expandable wall technology. The system affords the ability to install a retention ring through the sole (or upper) of a shoe similar to a grommet installation. This type of installation is done post-production through the sole of the shoe. The retention ring is inserted through the sole of the shoe. After the retention ring is installed, a force applicator (arch plate) is installed under the sock liner under the shoe. The entire sock liner may also be removed and replaced with an insert that incorporates a force applicator connected to padding to form the inner inset of the shoe. The force applicator is designed such that it can receive the telescoping element. The area contacting the telescoping element may be thicker or shaped such that it receives a portion of the telescoping element to maintain a connection or position between the two.

Alternatively, the retention ring can also be formed in the shoe sole during manufacture, thus creating a passageway through the sole of the shoe to allow the telescoping element the ability to interact with a force applicator in the arch area of the foot. As the telescoping element is translated through the sole of the shoe the translation of the force applicator upward or downward correspondingly results in increasing or decreasing the arch height of the shoe.

In accordance with various embodiments of the present invention, the system can be installed in other areas of the shoe. The invention assembly may be installed under the tripod area of the foot structure (under 1st metatarsal head, under 5th metatarsal head and under the calcaneous). This type of application allows the user to add support under any one of the support points of the foot, allowing the user to change the angle the foot and ankle are interacting with the ground. As such the foot may be tilted inward, outward or tipped up or down. The ability to wedge the foot is accomplished the same way. In Sport footwear such as hockey skates or hiking boots the invention may be installed superior to the ankle to stabilize unwanted medial—lateral movement. The device is installed in between the outer and inner portions of the boot and act to expand the inner portion of the boot inward to tighten the fit of the shoe/sport boot. The expansion of the upper portion of the footwear helps to stabilize the foot and ankle complex. Additionally, the installation of the device into the tongue of the footwear can be used to secure the foot to the shoe. In various embodiments, the retention ring is installed in the outer portion of the tongue of the shoe. The force applicator is integrated within the tongue and connected to the padding and inner lining of the tongue. In accordance with various embodiments of the present invention, the system functions to tighten the shoe over the dorsum of the user's foot, which eliminates the need for laces in the shoe. The expandable wall system can be configured in all of the embodiments described for the device referencing the prosthetic socket application.

Arch Support: With Adjustable Arch height. Foot orthotics are the foundation of orthopedic bracing. The orthopedic alignment of the foot is typically managed by foam or plastic arch supports that do not offer the ability to adjust the arch height of the device. Adjustments are often are made after viewing the patient walking on the device to help improve the foot alignment in a weighted position (typically by adding material to the arch area). In accordance with various embodiments of the present invention, a system is provided with an arch support with a top layer with an integrated force applicator and an outer frame or bottom layer where the retention ring is mounted. The telescoping element is inserted though the bottom frame into the retention ring and interacts or is connected to the force applicator. The user may then increase or decrease arch height by adjusting the telescoping element. The expandable wall system can be configured in all of the embodiments described for the device referencing the prosthetic socket application.

The detailed description of various embodiments herein makes reference to the accompanying figures, which show the exemplary embodiments by way of illustration. While these exemplary embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment. Definitions for terms that may be used throughout this disclosure are exemplary and non-exclusive. The terms used in this disclosure may have alternate meanings or definitions consistent with the disclosure, and the present disclosure is not limited to any particular definition or interpretation of any particular term.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Although the disclosure includes a method, it is contemplated that it may be embodied as computer program instructions on a tangible computer-readable carrier, such as a magnetic or optical memory or a magnetic or optical disk. All structural, chemical, and functional equivalents to the elements of the above-described various embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An expandable wall prosthetic socket for a residual limb, comprising:
a fixed retention ring installed into a side of a wall of the socket; and
a telescoping element, wherein the telescoping element interacts with the fixed retention ring to allow the telescoping element to translate inwardly and outwardly through the socket wall, such that the telescoping element is producing a load on the residual limb.

2. The expandable wall prosthetic socket of claim 1, wherein the fixed retention ring comprises a fixed retention plate.

3. The expandable wall prosthetic socket of claim 1, further comprising a force applicator connected to the socket wall, wherein when the telescoping element translates inwardly, the telescoping element makes contact with the force applicator such that the force applicator produces the load on the residual limb.

4. The expandable wall prosthetic socket of claim 3, wherein the force applicator further comprises a specific geometry such that the telescoping element makes contact with the specific geometry of the force applicator.

5. The expandable wall prosthetic socket of claim 3, wherein the telescoping element comprises a plurality of interconnected elements.

6. The expandable wall prosthetic socket of claim 3, wherein the force applicator is attached onto a prosthetic liner.

7. The expandable wall prosthetic socket of claim 1, further comprising a force applicator connected to the socket wall, wherein the telescoping element is anchored to the force applicator, wherein when the telescoping element translates inwardly, the force applicator produces the load on the residual limb.

8. The expandable wall prosthetic socket of claim 1, wherein the telescoping element comprises a plurality of telescoping elements.

9. The expandable wall prosthetic socket of claim 1, wherein the telescoping element comprises a plurality of telescoping elements, and wherein the expandable wall prosthetic socket further comprises a plurality of force applicators connected to the socket wall, wherein when one of the plurality of telescoping elements translates inwardly, the telescoping element makes contact with one of the plurality of force applicators such that the force applicator produces the load on the residual limb.

10. The expandable wall prosthetic socket of claim 1, wherein the expandable wall prosthetic socket is a 3D printed prosthetic socket, and wherein the fixed retention ring comprises a 3D printed aperture in a wall of the 3D printed prosthetic socket.

11. The expandable wall prosthetic socket of claim 3, wherein:
the expandable wall prosthetic socket is a 3D printed prosthetic socket;
the fixed retention ring comprises a 3D printed aperture in a wall of the 3D printed prosthetic socket;
the telescoping element is a 3D printed telescoping element; and
the force applicator is a 3D printed force applicator.

12. An expandable wall prosthetic socket for a residual limb, comprising:
a retention ring installed into a side of a wall of the socket, wherein the retention ring comprises a rotating segment; and
a telescoping element, wherein the telescoping element interacts with the retention ring to allow the telescoping element to translate inwardly and outwardly through the socket wall, such that the telescoping element is producing a load on the residual limb.

13. The expandable wall prosthetic socket of claim 12, further comprising a force applicator connected to the telescoping element, wherein when the telescoping element translates inwardly, the force applicator produces the load on the residual limb.

14. The expandable wall prosthetic socket of claim 13, wherein the telescoping element comprises a plurality of interconnected elements.

15. The expandable wall prosthetic socket of claim 13, wherein the force applicator is attached onto a prosthetic liner.

16. The expandable wall prosthetic socket of claim 12, wherein the telescoping element comprises a plurality of telescoping elements.

17. The expandable wall prosthetic socket of claim 12, wherein the telescoping element comprises a plurality of telescoping elements, and wherein the expandable wall prosthetic socket further comprises a plurality of force applicators, wherein each of the plurality of force applicators is connected to a different one of the plurality of telescoping elements, wherein when one of the plurality of telescoping elements translates inwardly, the connected force applicator produces the load on the residual limb.

18. An expandable wall prosthetic socket for a residual limb, comprising:
an applicator having a back plate and a front plate;
wherein the back plate comprises a retention ring, and wherein the back plate is mounted to a wall of the socket;
a telescoping element, wherein the telescoping element interacts with the retention ring to allow the telescoping element to translate inwardly and outwardly through the socket wall;
wherein the front plate comprises a force applicator, wherein when the telescoping element translates inwardly, the telescoping element makes contact with the force applicator such that the force applicator produces the load on the residual limb.
